(19) United States
(12) Patent Application Publication
(10) Pub. No.: US 2017/0027738 A1
(43) Pub. Date: Feb. 2, 2017

(54) A MucoAdhesive Tongue Strip Used As A Method to Prevent Snoring and Sleep Apnea During Sleep

(71) Applicant: Henry T Van, Tustin, CA (US)

(72) Inventor: Henry T Van, Tustin, CA (US)

(21) Appl. No.: 14/588,650

(22) Filed: Jan. 2, 2015

(51) Int. Cl. A61F 5/56; A61M 31/00

(52) U.S. Cl. CPC A61F 5/566; A61M 31/00

(57) ABSTRACT

A comfortable Mucoadhesive Tongue Strip that is applied to the tip of the tongue to keep the tongue in its restive position so as to reduce or eliminate the harmful effects of obstructive sleep apnea and disruptive snoring during sleep.

This invention relates to increasing the size of the air passageway through the oro- and naso pharynx during sleep by means of holding the tip of the tongue forward of the dental arches, and thus significantly reducing and or eliminating the harmful effects of obstructive sleep apnea and snoring.

### BACKGROUND OF INVENTION

Snoring is caused by vibration of respiratory structures due to obstructed air movement during breathing while sleeping. With increasing age, weight gain, and during periods of unconsciousness, some muscle tone is lost, thereby allowing one or more of the tongue, the pharyngeal flaps, the soft palate, the uvula, and the posterior pharyngeal wall to vibrate as tidal air flows therepast.

In heavy snorers, in addition to the noise, the increase pharyngeal resistance limits flow on breathing in at various levels, in particular in the region of the soft palate and the base of the tongue which are pressed against the posterior pharyngeal wall.

Sleep apnea gives rise to the condition where the tongue relaxes and contributes to the blocking of the air passageway in the pharynx for breathing from the nasal and oro opening. The most common type of sleep apnea is Obstructive Sleep Apnea. Obstructive Sleep Apnea (OSA) is defined as cessation in breathing for at least 10 seconds or longer during sleep and can occur hundreds of times during the night. In the supine sleep position, the tongue tissue relaxes during sleep and has tendency to fall back towards the pharynx, and cause it to collapse and block the upper airway. As a result, a reduction in blood oxygen saturation is present. The brain responds by rousing the body from sleep so as to restore normal breathing. This chronic sleep deprivation results in daytime sleepiness, drowsiness, headaches, slow reflexes, poor concentration, and an increased risk of accidents. Sleep apnea can also lead to serious health problems over time, including diabetes, high blood pressure, heart disease, stroke, and weight gain.

Treatment options for persons suffering from snoring and obstructive sleep apnea vary a great deal, from conservative treatment to invasive surgical treatment. Treatment for snoring includes positive airway pressure devices, dental appliances, and surgeries that are customized to best address each individual needs. The Continuous Positive Airway Pressure device (CPAP) is a machine that keeps the patient's airway open during sleep by delivering a continuous flow of pressurized air into the throat. Another option is an Oral Appliance Therapy (OAT) which is custom made mouthpiece that shifts the lower jaw forward, thereby opening the airway.

Other treatments include invasive surgeries that aim at opening the airway at different levels where obstructions are commonly encountered. These surgeries are individualized to address all anatomical areas of obstructions for each patient and may include surgeries such as maxillomandibular advancement (jaw surgery), hyoid suspension, base of tongue advancement surgeries, pharyngeal surgeries, and nasal surgeries (deviated septum and or enlarged turbinates).

Lastly, many types of tongue holding devices are known from prior arts. For example, metallic or hard plastic clips are disclosed in the art, e.g., in U.S. Pat. No. 4,198,967—Dror and U.S. Pat. No. 3,809,094—Crook. However, these devices risk pain and injury to the tongue, and are particularly unsuited to self-administration. A less traumatic device designed for self-administration and for extended periods of use (i.e., overnight) is disclosed in U.S. Pat. Nos. 4,169,473 and 4,304,277, both to Samelson. The device disclosed in the Samelson patents evacuates air from a tongue holder and uses an imperforate structure in a device that is positioned by holding both dental arches in a locked position. Such a device, however, is detrimental to the normal bite relationship of the dental arches since it distorts the relationship of the upper and lower jaws.

There remains, however, a need for a simple and reliable device that can comfortably restrain the tongue in a predetermined position. It is therefore an object of the present invention is to provide a simple, effective, inexpensive, and comfortable means for positioning the tongue in its restive position during sleep for OSA and snoring.

### BRIEF SUMMARY OF THE INVENTION

There has been many Prior Arts on the inventions of tongue locking devices to prevent OSA and snoring. The objective of this invention relates to increasing the size of the air passageway through the oro- and naso pharynx during sleep by means of holding the tip of the tongue forward of the dental arches, and thus significantly reducing and or eliminating the harmful effects of obstructive sleep apnea and snoring. By means of controlling the tongue positioning during sleep, a comfortable Mucoadhesive Tongue Strip 10 will be introduced.

The Mucoadhesive Tongue Strip can be of any color, sizes, shapes, thickness, and substrates as long as it fits comfortably on a person's tongue in its restive positioning during sleep. The Mucoadhesive Tongue Strip is a two part components that includes the pressure sensitive top and bottom sided mucoadhesive strips with said substrate (ie: hydrogel, silicone gel, gelatin based).

Of the Mucoadhesive Tongue Strip, the user applies the Pressure Sensitive Bottom Side Mucoadhesive Strip to the tongue. The Pressure Sensitive Top Side Mucoadhesive Strip is applied to the user's hard palate. As the tongue is held forwardly of its restive position behind the dental arches/hard palate, the size of the air passageway is normalized during sleep. This allows for comfortable and relaxed breathing that will eliminate OSA and snoring.

Prior Arts with tongue locking devices are uncomfortable and painful to wear on the tongue. Due to customization of the devices, it can be very costly to maintain and required daily cleansing for proper hygiene. With the Mucoadhesive Tongue Strip, it can be disposed after daily usage. There is no mess or cleaning required.

### BRIEF DESCRIPTION OF THE DRAWINGS

FIG. 1. Is an illustrative view showing the different shapes and sizes of the mucoadhesive strip to be used on the tongue

FIG. 2. Is an illustrative side, cross-sectional, exploded view of the mucoadhesive tongue strip.

FIG. 3. Is an illustrative close up view of the oral cavity wearing the mucoadhesive tongue strip.

FIG. 4. Is an illustrative cross sectioned side view of the human oro pharynx anatomy at its supine sleep position while wearing the mucoadhesive tongue strip during sleep.

FIG. 5. Is an illustrative cross sectioned side view of the human oro-pharynx anatomy at its supine sleep position during sleep with obstructive sleep apnea.

### DETAILED DESCRIPTION OF THE INVENTION

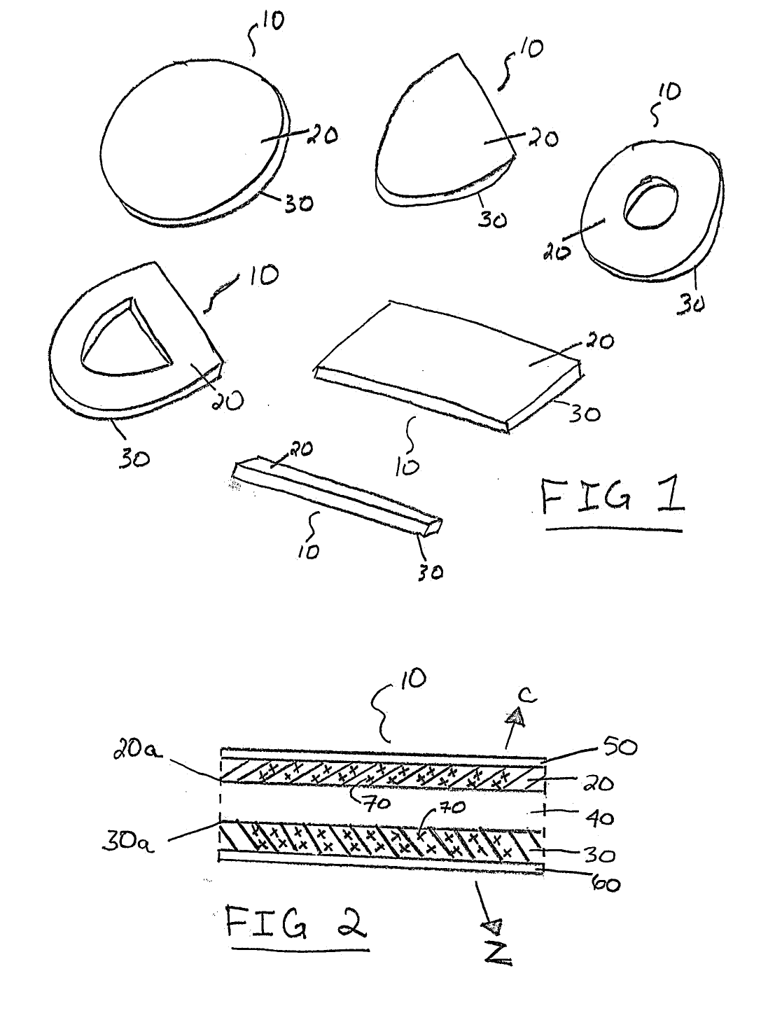

Referring now to FIG. 1, Depicts the different sizes and shapes of the Mucoadhesive Tongue Strip 10.
a) The exact sizes, shapes, thickness, and colors can vary as long as the Mucoadhesive Tongue Strip 10 can fit comfortably to the user's tongue during sleep.
b) The Mucoadhesive Tongue Strip 10 can be made of various substrates 40, (ie. hydrogel, silicone gel, gelatin based (gummy bear), polyurethane sheet, polyethylene film, wax, or medical tape.).
c) Of the Mucoadhesive Tongue Strip 10, comprises of Pressure Sensitive Bottom Side Mucoadhesive Strip 30 and Pressure Sensitive Top Side Mucoadhesive Strip 20.

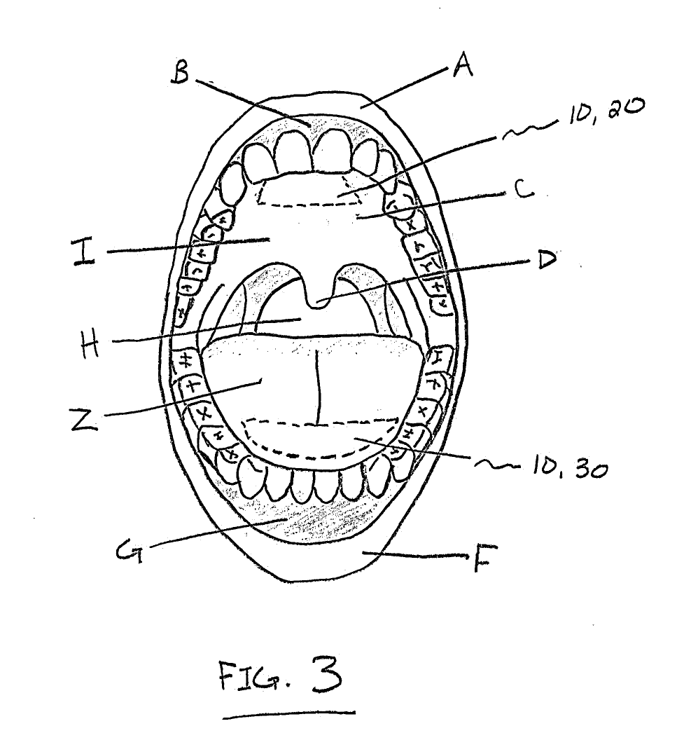

FIG. 2 depicts an embodiment illustrative side, cross sectional, exploded view of the Adhesive Tongue Strip.
1. The embodiments of an Mucoadhesive Tongue Strip 10 comprising of:
   a) Substrate 40 (one example: Hydrogel)
   b) Double Sided Mucoadhesive Strips
      i. Pressure Sensitive Top Side Mucoadhesive Strip 20
      ii. Pressure Sensitive Bottom Side Mucoadhesive Strip 30
   c) Polyethylene or foil sheet backing on both sides of the mucoadhesive strips 50 and 60
2. The Mucoadhesive Tongue Strip 10, wherein can be of various substrates 40 (ie: hydrogel, silicone gel, gelatin based (gummy bear), polyurethane sheet, medical tape). The preferred substrate will be hydrogel or silicone gel since this substrate can easily conforms to the users tongue and rigid hard palate.
3. The Mucoadhesive Tongue Strip 10 wherein is a comfortable, safe, non-toxic, hypoallergenic, biodegradable, and biocompatible with double sided mucoadhesive tongue strip 10 that is applied at the tip of the tongue Z on one side and hard palate C on the other side
4. The Mucoadhesive Tongue Strip 10, wherein the embodiment comprising of the top side and bottom side mucoadhesive strips. Pressure Sensitive Top Side Mucoadhesive Strip 20 and Pressure Sensitive Bottom Side Mucoadhesive Strip 30 are bonded with an adhesive to each side of the substrate 40 (top substrate 20a and bottom substrate 30a) with acrylate adhesives, thermoplastics “hot melt” adhesives, two sided adhesive tape, elastomer based adhesives, or acrylic adhesives type composition.
5. The Mucoadhesive Tongue Strip 10, wherein is a pressure sensitive mucoadhesive strips 20 and 30 formulated for immediate removal or erode over a period of wear time with a gentle removal force. In this instance, the preferred pressure sensitive mucoadhesive strength is between 6-8 hours of usage.
6. The Mucoadhesive Tongue Strip 10, wherein is a specialty formulated pressure sensitive mucoadhesive strips 20 and 30 for bonding to wet mucosal tissues designed for oral use
7. The Mucoadhesive Tongue Strip 10, wherein may include a layer of specialty formulated mucoadhesive of aromatic medicate substances 70 comprise of camphor, eucalyptus oil, peppermint oil, menthol, methylsalicylate, bornyl acetate, lavender oil, citrus, an antihistamine, a decongestant, or combinations thereof.
8. The Mucoadhesive Tongue Strip 10, wherein consists of Pressure Sensitive Top Side Mucoadhesive Strip 20 and Pressure Sensitive Bottom Mucoadhesive Side Strip 30 that is reversible or interchangeable on either sides (top/bottom and bottom/top) when applying to the tongue Z and hard palate C.
9. The Mucoadhesive Tongue Strip 10, wherein includes a backing layer of polyethylene or foil sheets 50 and 60 that covers the Pressure Sensitive Top Side Strip 20 and Pressure Sensitive Bottom Side Strip 30 when not in use. Once the adhesive tongue strip 10 is ready for use, the user simply removes the backing layer foil sheets 50 and 60 from the adhesive tongue strip 10 on each side of the mucoadhesive strips 20 and 30.
10. The Mucoadhesive Tongue Strip 10, wherein can be of different sizes, shapes, and thickness that are anatomical to the tongue shape for comfort
11. The Mucoadhesive Tongue Strip 10 does not require any type of dental appliance to be used. It is used as a standalone strip.
12. A method of treating patients experiencing obstructive sleep apnea and snoring comprising:
   a) A Mucoadhesive Tongue Strip 10 with substrate 40 and the Pressure Sensitive Top Side Mucoadhesive Strip 20 and Pressure Sensitive Bottom Side Mucoadhesive Strip 30.
   b) Adhesively applying the Pressure Sensitive Bottom Side Mucoadhesive Strip 30 to the user's tip of tongue Z
   c) Adhesively applying the Pressure Sensitive Top Side Mucoadhesive Strip 20 to the user's hard palate C
   d) While mouth is closed and tongue Z is in its restive position, a person may freely breathe with mouth closed or opened during sleep

Referring now to FIG. 3. Depicts the anatomical structure of the oral cavity when the user applies the Mucoadhesive Tongue Strip 10 in its present invention. The oral mouth includes an upper jaw B, a lower jaw G, the tongue Z, hard palate C and soft palate I, the uvula D, and the posterior pharyngeal wall (pharynx) H.
A: Upper Lip
B: Upper Jaw Gingiva (Gum)
C: Hard Palate
D: Uvula
Z: Tongue
30: Mucoadhesive Tongue Strip 10 placed on the tongue on one side of the mucoadhesive strip
F: Lower Lip
G: Lower Jaw Gingiva (Gum)
H: Pharyngeal Wall (Pharynx)
I: Soft Palate
20: Mucoadhesive Tongue Strip 10 placed on the hard palate on the one side of the mucoadhesive strip

With reference to the FIG. 3, the user applies the Mucoadhesive Tongue Strip 10 on the tip of the tongue Z and the hard palate C. The special formulated safe, non toxic, biodegradable mucoadhesive strips will safely adhere to the wet mucosal tissues of the tongue Z and the hard palate C. The sizes, shapes, and substrate 40 type can vary so long as it fits anatomically to the user's tongue Z in a comfortable restive position.
1) A Mucoadhesive Tongue Strip 10 with substrate 40 and the Pressure Sensitive Top and Bottom Side Layers Mucoadhesive 20 and 30 respectively.
   a) Adhesively applying the Pressure Sensitive Bottom Side Layer Mucoadhesive 30 to the user's tip of tongue Z
   b) Adhesively applying the Pressure Sensitive Top Side Layer Mucoadhesive 20 to the user's hard palate C
   c) While mouth is closed and tongue Z is in its restive position, a person may freely breathe with mouth closed or opened during sleep

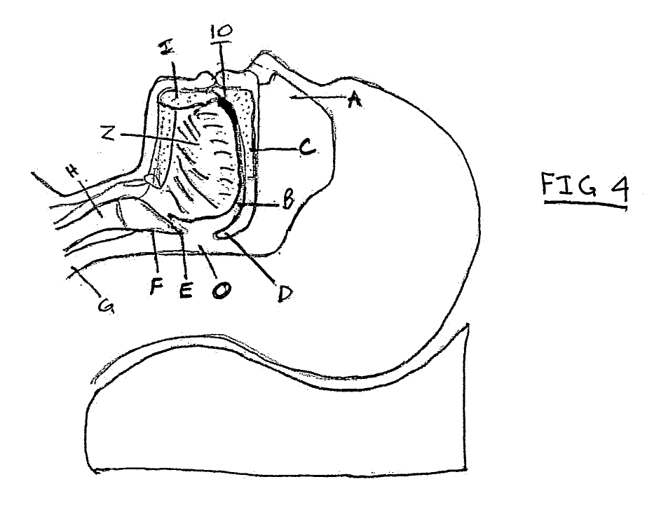

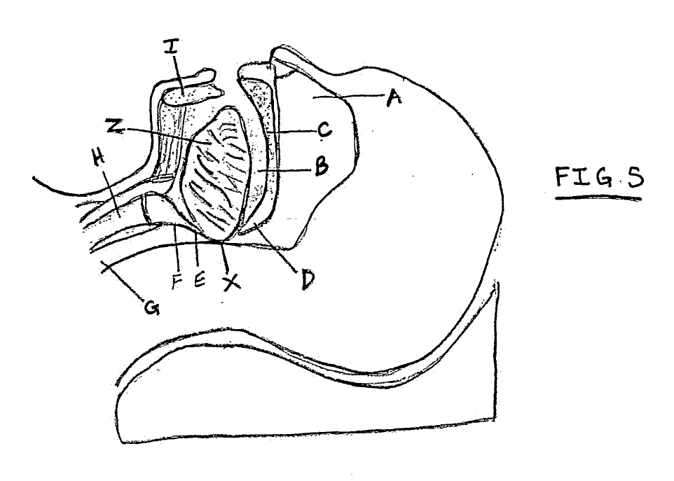

Referring now to FIG. 4. Depicts an embodiment illustrative cross sectioned side view of the user oro-pharynx anatomy at its supine sleep position while wearing the Mucoadhesive Tongue Strip 10 during sleep. The Adhesive Tongue Strip 10 holds the tongue Z in its position and prevents tongue Z from falling back towards the pharnx F and prevents it to collapse and block the upper airway O. The user can breathe freely through the oro-naso pharynx opening as a result of the airway passage O.
10: Tongue Adhesive Strip depict in dark shade on the tongue
A: Nasal Cavity
B: Oral Cavity
C: Hard Palate
D: Uvula
O: Naso and Oro-Pharynx Open Air Passageway
E: Epiglottis
F: Larynx opening to Pharynx
G: Esophagus
H: Lamyx
I: Lower Jaw
Z: Tongue

Referring now to FIG. 5. Depicts an illustrative cross sectioned side view of the user oro-pharynx anatomy at its supine sleep position during sleep. The illustrative shows the tongue Z tissue relaxes during sleep and has tendency to fall back towards the pharnx F, and cause it to collapse and block the upper airway X. The collapse of the airway X causes cessation in breathing that will result in OSA.
A: Nasal Cavity
B: Oral Cavity
C: Hard Palate
D: Uvula
X: Naso and Oro-Pharynx Obstruction
E: Epiglottis
F: Larynx opening to Pharynx
G: Esophagus
H: Larynx
I: Lower Jaw
Z: Tongue

What is claimed and desired to be secured by Letters Patent of the United States is:

1. What is claimed is an Mucoadhesive Tongue Strip of claim 1, comprising of:
a) Substrate (ie. example: Hydrogel, Silicone Gel, Gelatin Based)
b) Double Sided Mucoadhesive Strips

2. The Mucoadhesive Tongue Strip of claim 1, wherein consists of double sided pressure sensitive mucoadhesive strips on said substrate that is interchangeable or reversible on either side (top/bottom and bottom/top) when applying to the tongue and or hard palate
a) Comprised of Pressure Sensitive Top Side Mucoadhesive Strip that is applied to the user's hard palate
b) Comprised of a Pressure Sensitive Bottom Side Mucoadhesive Strip that is applied to the user's tongue

3. The Mucoadhesive Tongue Strip of claim 1, wherein is a specialty formulated pressure sensitive mucoadhesive for bonding to wet mucosal tissues designed for oral use

4. The Mucoadhesive Tongue Strip of claim 1, wherein is a pressure sensitive mucoadhesive formulated for immediate removal or erode over a period of wear time with a gentle removal force.

5. The Mucoadhesive Tongue Strip of claim 1, wherein is a pressure sensitive adhesive based on strength of formulation

6. The Mucoadhesive Tongue Strip of claim 1, wherein may have an optional layer of aromatic medicated substance which comprises of: camphor, eucalyptus oil, peppermint oil, menthol, citrus, antihistamine, decongestant, chemical variations or combinations thereof within the pressure sensitive mucoadhesive strips

7. A method of applying the Mucoadhesive Tongue Strip during sleep comprising:
a) A comfortable, safe, non-toxic, biodegradable mucoadhesive tongue strip made of said substrate that is applied at the tip of the tongue on the bottom side of the pressure sensitive mucoadhesive strip and the top side pressure sensitive mucoadhesive strip to the hard palate so as to hold the tongue forward in a restive position behind the dental arches during sleep.
b) Having the tongue “secured” in its restive position will prevent the tongue from blocking the pharyngeal and oral airflow passageway during sleep. The dimension of air passage through the oro- and naso-pharnx, around the soft palate, the uvula, and the posterior pharyngeal wall is increased.
